(19) United States
(12) Patent Application Publication
(10) Pub. No.: US 2025/0014295 A1
(43) Pub. Date: Jan. 9, 2025

(54) SYSTEMS AND METHODS FOR DETERMINING MATERIAL PROPERTY VALUES FOR A THREE-DIMENSIONAL VIRTUAL MODEL OF AN ANATOMICAL OBJECT

(71) Applicant: Intuitive Surgical Operations, Inc., Sunnyvale, CA (US)

(72) Inventors: Pourya Shirazian, Menlo Park, CA (US); Kenneth K. Lee, Oakton, VA (US)

(21) Appl. No.: 18/704,649

(22) PCT Filed: Dec. 15, 2022
(86) PCT No.: PCT/US2022/053046

§ 371 Date: Apr. 25, 2024

Related U.S. Application Data
(60) Provisional application No. 63/290,823, filed on Dec. 17, 2021.

(51) Int. Cl. G06T 19/20; G06T 7/593; G16H 50/50

(52) U.S. Cl. CPC G06T 19/20; G06T 7/593; G16H 50/50; G06T 2207/10012; G06T 2207/10016; G06T 2207/30004; G06T 2210/41; G06T 2219/2021

(57) ABSTRACT

An illustrative virtual model simulation system may be configured to identify surface points on an anatomical object as depicted in one or more video images, determine a set of displacement values representative of a displacement of one or more of the surface points of the anatomical object caused by a physical force being applied by a physical tool to the anatomical object, and define, based on the set of displacement values and data representative of the physical force, one or more material property values for a three-dimensional (3D) virtual model of the anatomical object.

### RELATED APPLICATIONS

The present application claims priority to U.S. Provisional Patent Application No. 63/290,823, filed Dec. 17, 2021, the contents of which is hereby incorporated by reference in its entirety.

### BACKGROUND INFORMATION

A system used during a simulation session may present a three-dimensional (3D) virtual model in which a user may interact with the 3D virtual model. For example, the system may present a 3D virtual model of an anatomical object that may be manipulated by a user using one or more virtual tools.

In some instances, it may be desirable for the 3D virtual model to simulate physical interaction with the anatomical object. For example, it may be desirable to present a 3D virtual model of an anatomical object that deforms in substantially the same way as a real anatomical object in response to manipulation by the user.

To this end, it may be desirable for the 3D virtual model to include material property values representative of a material property of the anatomical object and that affect how the 3D virtual model deforms to more closely mimic the physical deformation of the anatomical object. Unfortunately, determining such material property values may be a complex and/or time-consuming process and may not result in a physically accurate 3D virtual model. For example, the material property values may fail to account for variations in the anatomical object (e.g., variations in material properties at different locations along the anatomical object and/or variations in material properties of the anatomical object from patient to patient) and/or for external objects proximate to the anatomical object that may affect how the anatomical object deforms. This may result in a 3D virtual model that fails to realistically mimic deformation of the real anatomical object being simulated.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An illustrative system includes a memory storing instructions and a processor communicatively coupled to the memory and configured to execute the instructions to: identify surface points on an anatomical object as depicted in one or more video images; determine a set of displacement values representative of a displacement of one or more of the surface points of the anatomical object caused by a physical force being applied by a physical tool to the anatomical object; and define, based on the set of displacement values and data representative of the physical force, one or more material property values for a 3D virtual model of the anatomical object, the one or more material property values representative of a material property of the anatomical object.

An illustrative method includes identifying, by at least one computing device, surface points on an anatomical object as depicted in one or more video images; determining, by the at least one computing device, a set of displacement values representative of a displacement of one or more of the surface points of the anatomical object caused by a physical force being applied by a physical tool to the anatomical object; and defining, by the at least one computing device based on the set of displacement values and data representative of the physical force, one or more material property values for a 3D virtual model of the anatomical object, the one or more material property values representative of a material property of the anatomical object.

An illustrative non-transitory computer-readable medium storing instructions that, when executed, direct a processor of a computing device to: identify surface points on an anatomical object as depicted in one or more video images; determine a set of displacement values representative of a displacement of one or more of the surface points of the anatomical object caused by a physical force being applied by a physical tool to the anatomical object; and define, based on the set of displacement values and data representative of the physical force, one or more material property values for a 3D virtual model of the anatomical object, the one or more material property values representative of a material property of the anatomical object.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative implementation including a virtual model simulation system.

FIG. 2 shows another illustrative implementation including a virtual model simulation system.

FIG. 3 shows an illustrative method of operating the virtual model simulation system of FIG. 2.

FIG. 4A shows an illustrative implementation of determining a set of displacement values of the method of FIG. 3.

FIG. 4B shows another illustrative implementation of determining a set of displacement values of the method of FIG. 3.

FIG. 5 shows an enlarged partial view of the illustrative implementation of FIG. 4B.

FIG. 6 shows another illustrative method of operating the virtual model simulation system of FIG. 2.

FIG. 7A shows an illustrative implementation of determining an additional set of displacement values of the method of FIG. 6.

FIG. 7B shows another illustrative implementation of determining an additional set of displacement values of the method of FIG. 6.

FIG. 8 shows an enlarged partial view of the illustrative implementation of FIG. 7B.

FIG. 9 shows an illustrative computer-assisted medical system that may incorporate the virtual model simulation system of FIG. 2.

FIG. 10 shows an illustrative computing system according to principles described herein.

### DETAILED DESCRIPTION

An illustrative virtual model simulation system may be configured to define one or more material property values, based on physical manipulation of an anatomical object, for a 3D virtual model of the anatomical object.

For example, the virtual model simulation system may be configured to identify surface points on an anatomical object as depicted in one or more video images, determine a set of displacement values representative of a displacement of one or more of the surface points of the anatomical object caused by a physical force being applied by a physical tool to the anatomical object, and determine, based on the set of displacement values and data representative of the physical force, one or more material property values of a 3D virtual model of the anatomical object. The one or more material property values may be representative of a material property of the anatomical object. In some embodiments, the one or more material property values may define how the 3D virtual model deforms in response to manipulation of the 3D virtual model.

As another example, the virtual model simulation system may be configured to perform a registration of the 3D virtual model with a 3D model derived from one or more video images depicting the anatomical object. The virtual model simulation system may further be configured to identify, based on the registration, surface vertices on a surface of the 3D virtual model that correspond to the surface points on the anatomical object, the material properties of the 3D virtual model affecting how the surface vertices are displaced in response to manipulation of the 3D virtual model, apply, to the 3D virtual model, a virtual force that simulates the physical force, and determine an additional set of displacement values representative of a displacement of one or more of the surface vertices of the 3D virtual model caused by the virtual force. The virtual model simulation system may be configured to adjust the one or more material property values until the additional set of displacement values is within a threshold amount of the set of displacement values representative of the displacement of one or more of the surface points of the anatomical object.

The embodiments described herein may result in improved 3D virtual model simulation of anatomical objects compared to conventional techniques that are not based on physical manipulation of the anatomical object, as well as provide other benefits as described herein. For example, by basing the determination of the material property values for a 3D virtual model on physical manipulation of an anatomical object, the material property values for the 3D virtual model may be more accurately determined compared to conventional techniques that rely, for example, on published data that is not specific to a particular patient. As described herein, the determined material property values based on physical manipulation of the anatomical object may account for variations in the anatomical object and/or for external objects proximate to the anatomical object that may affect how the anatomical object deforms. This may result in a 3D virtual model that more closely mimics deformation of the real anatomical object being simulated. It may further allow for the material property values of the 3D virtual model to be customizable for a particular patient and/or group of patients (e.g., based on age, race, gender, location, type of anatomical object, etc.).

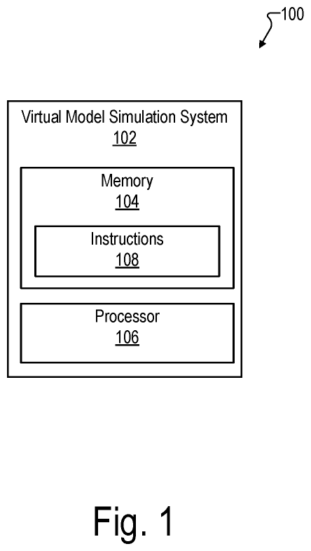

FIG. 1 shows an illustrative implementation 100 configured to define (e.g., establish, update, or otherwise determine) one or more material property values for a 3D virtual model of an anatomical object that are representative of a material property of the anatomical object. An anatomical object may include an object associated with a body (e.g., a body of a live animal, a human or animal cadaver, a portion of human or animal anatomy, tissue removed from human or animal anatomies, non-tissue work pieces, training models, etc.). In some implementations, the anatomical object may include a tissue associated with a body (e.g., an organ, soft tissue, connective tissue, etc.). A material property may include a property of the anatomical object that affects how the anatomical object deforms in response to a force applied to the anatomical object (e.g., a mass, stiffness, stress, strain, strength, hardness, elasticity, Young's modulus, Poisson Ratio, etc.). As shown, implementation 100 includes a virtual model simulation system 102. Implementation 100 may include additional or alternative components as may serve a particular implementation. In some examples, implementation 100 or certain components of implementation 100 may be implemented by a computer-assisted medical system.

Virtual model simulation system 102 may be implemented by one or more computing devices and/or computer resources (e.g., processors, memory devices, storage devices, etc.) as may serve a particular implementation. As shown, virtual model simulation system 102 may include, without limitation, a memory 104 and a processor 106 selectively and communicatively coupled to one another. Memory 104 and processor 106 may each include or be implemented by computer hardware that is configured to store and/or process computer software. Various other components of computer hardware and/or software not explicitly shown in FIG. 1 may also be included within virtual model simulation system 102. In some examples, memory 104 and/or processor 106 may be distributed between multiple devices and/or multiple locations as may serve a particular implementation.

Memory 104 may store and/or otherwise maintain executable data used by processor 106 to perform any of the functionality described herein. For example, memory 104 may store instructions 108 that may be executed by processor 106. Memory 104 may be implemented by one or more memory or storage devices, including any memory or storage devices described herein, that are configured to store data in a transitory or non-transitory manner. Instructions 108 may be executed by processor 106 to cause virtual model simulation system 102 to perform any of the functionality described herein. Instructions 108 may be implemented by any suitable application, software, code, and/or other executable data instance. Additionally, memory 104 may also maintain any other data accessed, managed, used, and/or transmitted by processor 106 in a particular implementation.

Processor 106 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), image signal processors, or the like. Using processor 106 (e.g., when processor 106 is directed to perform operations represented by instructions 108 stored in memory 104), virtual model simulation system 102 may perform various operations as described herein.

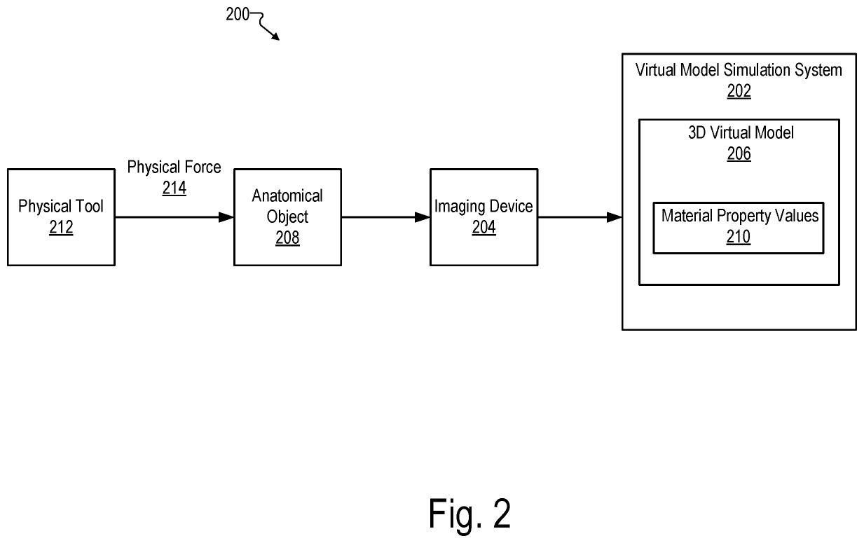

FIG. 2 shows another illustrative implementation 200 configured to define one or more material property values for a 3D virtual model of an anatomical object that are representative of a material property of the anatomical object. As shown, implementation 200 includes a virtual model simulation system 202 in communication with an imaging device 204. Implementation 200 may include additional or alternative components as may serve a particular implementation. In some examples, implementation 200 or certain components of implementation 200 may be implemented by a computer-assisted medical system.

Virtual model simulation system 202 may implement or be similar to virtual model simulation system 102. Virtual model simulation system 202 may be configured to present (e.g., by a display device) a 3D virtual model 206 of an anatomical object 208 having material property values 210 representative of material property of anatomical object 208.

Virtual model simulation system 202 may be configured to receive 3D virtual model 206 and/or generate 3D virtual model 206. In some implementations, virtual model simulation system 202 may be configured to generate 3D virtual model 206 based on one or more preoperative images of anatomical object 208. Some examples of generating a 3D virtual model are described in PCT Patent Publication No. WO/2019/005881, published on Jan. 3, 2019, and entitled “Unified Anisotropic Volume and Surface Mesh Storage,” the contents of which are hereby incorporated by reference in their entirety.

In instances where virtual model simulation system 202 is configured to generate 3D virtual model 206, virtual model simulation system 202 may be configured to receive a 3D image representation of anatomical object 208 from a scanning system (e.g., a Computerized Tomography (CT), a Magnetic Resonance Imaging (MRI), an Ultrasound, etc.). The 3D image representation of anatomical object 208 may include pixel data (e.g., pixels) arranged in a 3D grid.

Virtual model simulation system 202 may further be configured to derive a volumetric mesh model, based on the 3D image representation of anatomical object 208, to represent the physical anatomical object 208 during a simulation. For example, virtual model simulation system 202 may be configured to derive vertices arranged in a 3D grid configuration within the volumetric mesh model and associate the vertices with 3D locations that correspond to locations of the pixel data within the 3D image representation of anatomical object 208. In some implementations, virtual model simulation system 202 may be configured to separate surface vertices and internal vertices of the volumetric mesh model.

Virtual model simulation system 202 may further be configured to convert the volumetric mesh model to 3D virtual model 206 having material property values 210 (e.g., in a unified mesh format (UMF)). For example, virtual model simulation system 202 may associate a material property value 210 with each vertex of the volumetric mesh model to form 3D virtual model 206. In some implementations, one or more material property values 210 may be applied as a scalar value at each vertex such that material property values 210 affect how 3D virtual model 206 deforms in response to manipulation. For example, material property value 210 may include a Young's modulus in terms of stiffness.

Virtual model simulation system 202 may be configured to define material property values 210 based on physical manipulation of anatomical object 208 as depicted in one or more video images captured by imaging device 204. Imaging device 204 may be implemented by an endoscope or other suitable device to capture one or more video images (e.g., images and/or a sequence of image frames included in video captured by imaging device) of anatomical object 208 (e.g., during a medical procedure). For example, imaging device 204 may capture one or more video images of a physical tool 212 applying a physical force 214 to anatomical object 208 to manipulate anatomical object 208. Physical tool 212 may be implemented by a medical tool or other suitable device to apply physical force 214 to anatomical object 208 (e.g., a scalpel, scissors, forceps, a clamp, etc.).

To define material property values 210, virtual model simulation system 202 may be configured to identify surface points on anatomical object 208 as depicted in one or more video images captured by imaging device 204. Virtual model simulation system 202 may further be configured to determine a set of displacement values representative of a displacement of one or more of the surface points of anatomical object 208 (e.g., a change in location of the surface points in a direction, angle, velocity, acceleration, etc.) caused by physical force 214 being applied by physical tool 212 to anatomical object 208. Virtual model simulation system 202 may further be configured to determine, based on the set of displacement values and data representative of physical force 214, one or more material property values 210 of 3D virtual model 206 of anatomical object 208. For example, material property values 210 may be determined based on Hooke's Law (F=kx, where F is a force, k is a constant based on material properties, and x is a displacement). The one or more material property values 210 may be representative of a material property of anatomical object 208 and define how 3D virtual model 206 deforms in response to manipulation of 3D virtual model 206.

Data representative of physical force 214 may include a measure of physical force 214. In some implementations, the measure of physical force 214 may be obtained by a force sensor (not shown) communicatively coupled with physical tool 212. Additionally or alternatively, the measure of physical force 214 may be determined based on kinematic data representative of movement of physical tool 212 over time. For example, the kinematic data may be generated by a computer-assisted medical system communicatively coupled with physical tool 212. Data representative of physical force 214 may further include one or more physical properties of physical tool 212 (e.g., a type, a material, a size, etc.).

In some implementations, material property values 210 may be generated by virtual model simulation system 202 based substantially on the physical manipulation of anatomical object 208. Additionally or alternatively, virtual model simulation system 202 may adjust material property values 210 from baseline material property values based on the physical manipulation of anatomical object 208. For example, the baseline material property values may include known material properties associated with a type of anatomical object that have been published and/or determined from previous medical procedures. Accordingly, the material property values 210 determined by virtual model simulation system 202 may affect how 3D virtual model 206 deforms to more closely mimic how anatomical object 208 deforms in response to physical force 214. Virtual model simulation system 202 may be configured to present 3D virtual model 206 during a simulation session in which a user interacts with 3D virtual model 206 to simulate physical interaction with anatomical object 208.

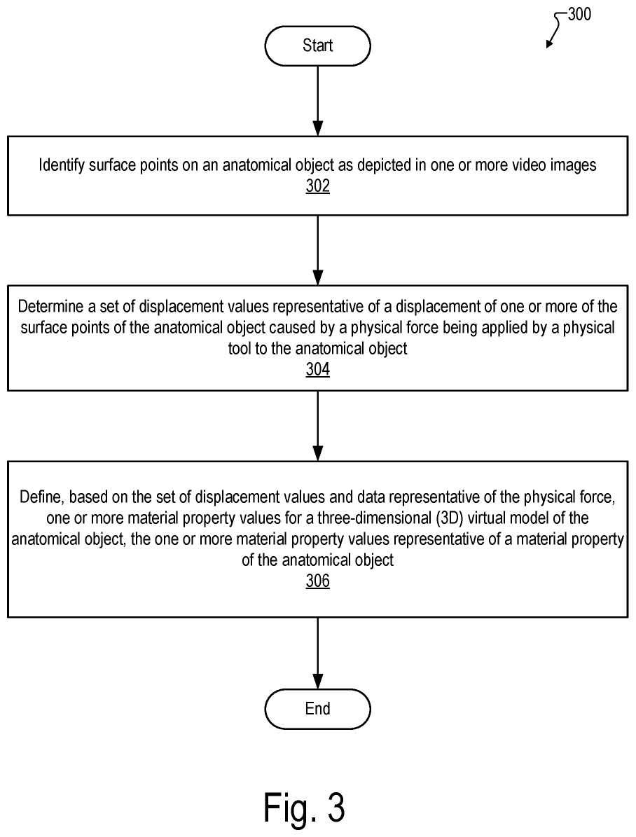

FIG. 3 shows an illustrative method 300 that may be performed by virtual model simulation system 202. While FIG. 3 illustrates exemplary operations according to one embodiment, other embodiments may omit, add to, reorder, and/or modify any of the operations shown in FIG. 3. Moreover, each of the operations depicted in FIG. 3 may be performed in any of the ways described herein.

As shown, virtual model simulation system 202 may, at operation 302, identify surface points on anatomical object 208 as depicted in one or more video images. Virtual model simulation system 202 may, at operation 304, determine a set of displacement values representative of a displacement of one or more of the surface points of anatomical object 208 caused by physical force 214 being applied by physical tool 212 to anatomical object 208. Virtual model simulation system 202 may, at operation 306, define, based on the set of displacement values and data representative of physical force 214, one or more material property values 210 for 3D virtual model 206 of anatomical object 208. In some examples, the one or more material property values 210 may define how 3D virtual model 206 deforms in response to manipulation of 3D virtual model 206.

In some implementations, the determining the set of displacement values may include generating a point cloud having a first plurality of nodes representative of the surface points on anatomical object 208 and tracking movement of the first plurality of nodes over time while physical force 214 is applied to anatomical object 208.

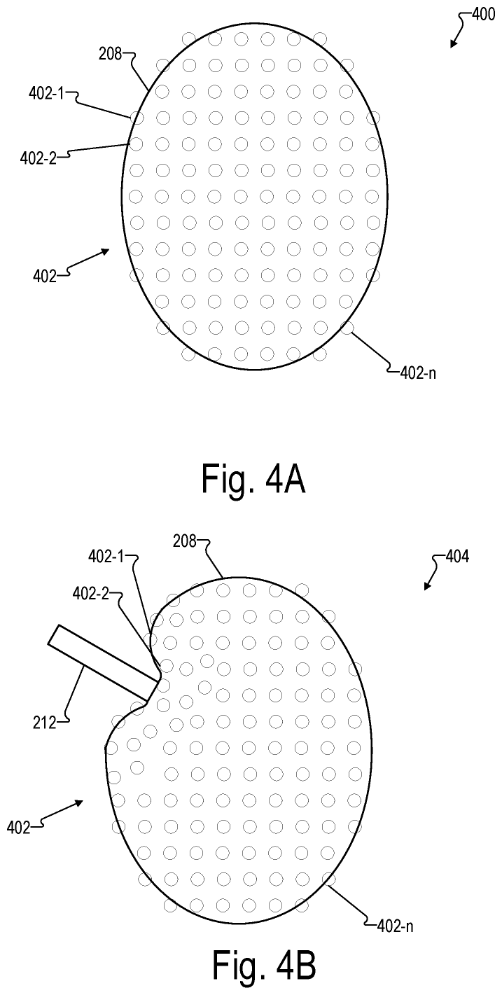

As an illustrative example, FIG. 4A shows an implementation 400 of a point cloud that may be generated by virtual model simulation system 202 having a first plurality of nodes 402 (e.g., nodes 402-1, 402-2 . . . 402-n) representative of the surface points on anatomical object 208. While FIG. 4A shows first plurality of nodes 402 positioned in a 3D grid configuration, virtual model simulation system 202 may be configured to generate any suitable number of nodes 402 in any suitable configuration.

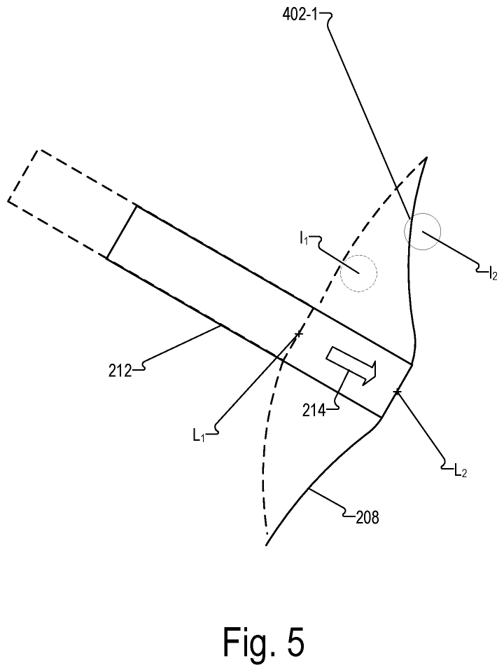

FIGS. 4B and 5 show an illustrative implementation 404 of tracking movement of nodes 402. For example, one or more nodes 402 may move in response to physical tool 212 applying physical force 214 to anatomical object 208. As depicted in FIG. 5, a tip of physical tool 212 may be moved from a first location L₀ to a second location L₂ relative to anatomical object 208 over an amount of time to apply physical force 214 to anatomical object 208. In response to physical force 214, anatomical object 208 may deform such that a first node 402-1 may move from a first location I₁ to a second location I₂ over the amount of time. Virtual model simulation system 202 may be configured to determine a displacement value representative of a displacement (e.g., a distance, direction, angle, velocity, acceleration, etc.) of first node 402-1 based on the movement of first node 402-1. Virtual model simulation system 202 may further be configured to determine a set of displacement values representative of a displacement of each node of the first plurality of nodes 402.

In some implementations, the tracking movement of nodes 402 may include determining a depth of nodes 402 (e.g., first node 402-1) relative to an initial position (e.g., location I₁) of nodes 402 prior to application of physical force 214. For example, the one or more video images captured by imaging device 204 may be configured to capture stereoscopic images of anatomical object 208 that may be processed in any suitable manner to determine the depth of nodes 402. Additionally or alternatively, a depth of nodes 402 may be determined by generating, based on depth measurements obtained by a depth sensor (not shown), a depth map of nodes 402. Still other suitable methods for determining a depth of nodes 402 may be used.

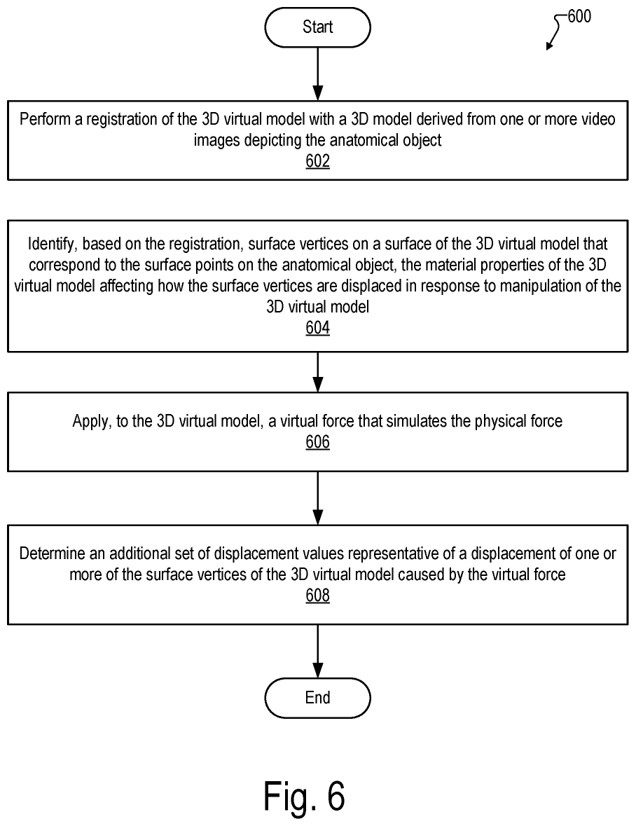

FIG. 6 shows another illustrative method 600 that may be performed by virtual model simulation system 202. While FIG. 6 illustrates exemplary operations according to one embodiment, other embodiments may omit, add to, reorder, and/or modify any of the operations shown in FIG. 6. Moreover, each of the operations depicted in FIG. 6 may be performed in any of the ways described herein.

As shown, virtual model simulation system 202 may, at operation 602, perform a registration of 3D virtual model 206 with a 3D model derived from the one or more video images depicting anatomical object 208 (e.g., by imaging device 204). Virtual model simulation system 202 may, at operation 604, identify, based on the registration, surface vertices on a surface of 3D virtual model 206 that correspond to the surface points on anatomical object 208, material properties values 210 of 3D virtual model 206 affecting how the surface vertices are displaced in response to manipulation of 3D virtual model 206. Virtual model simulation system 202 may, at operation 606, apply, to 3D virtual model 206, a virtual force that simulates physical force 214. Virtual model simulation system 202 may, at operation 608, determine an additional set of displacement values representative of a displacement of one or more of the surface vertices of 3D virtual model 206 (e.g., a change in location of the surface vertices in a direction, angle, velocity, acceleration, etc.) caused by the virtual force.

In some implementations, the determining the additional set of displacement values may include generating a point cloud having a second plurality of nodes representative of the surface vertices on 3D virtual model 206 and tracking movement of the second plurality of nodes over time while a virtual force is applied to 3D virtual model 206.

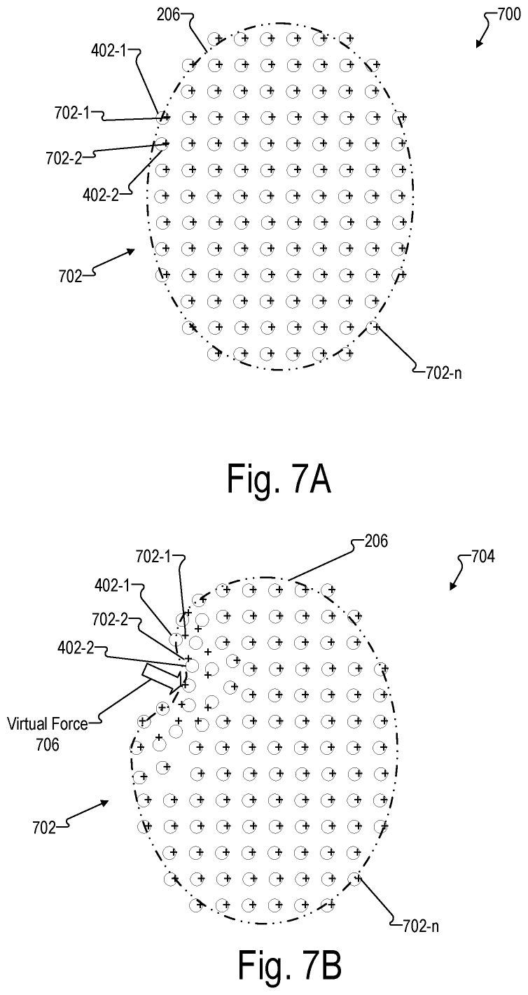

As an illustrative example, FIG. 7A show an implementation 700 of a point cloud that may be generated by virtual model simulation system 202 having a second plurality of nodes 702 (e.g., nodes 702-1, 702-2 . . . 702-n, each illustrated by a “+”) representative of the surface vertices on 3D virtual model 206. The 3D position of nodes 702 may correspond to the 3D location of nodes 402 of anatomical object 208. While FIG. 7A shows second plurality of nodes 702 positioned in a 3D grid configuration, virtual model simulation system 202 may be configured to generate any suitable number of nodes 702 in any suitable configuration.

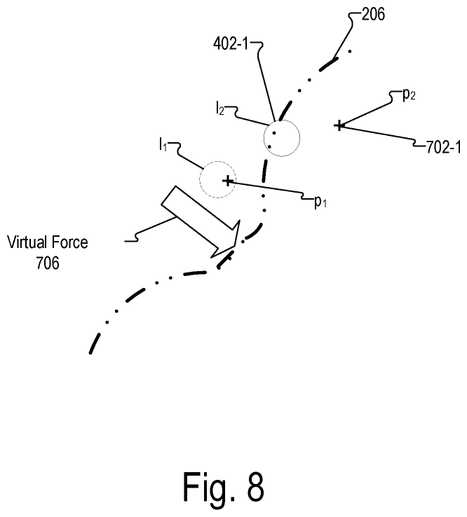

FIGS. 7B and 8 show an illustrative implementation 704 of tracking movement of nodes 702. For example, virtual model simulation system 202 may be configured to simulate application of physical force 214 that imparts physical deformation to anatomical object 208 by applying a virtual force 706 that corresponds to physical force 214 to 3D virtual model 206 that imparts virtual deformation to 3D virtual model 206. In some implementations, virtual model simulation system 202 may apply a measure of virtual force 706 that is similar to the measure of physical force 214. Additionally or alternatively, virtual model simulation system 202 may simulate a virtual tool of a similar type as physical tool 212 that moves in a similar manner as physical tool 212 over a similar amount of time.

During simulation, virtual model simulation system 202 may change or track the positions of nodes 702 within 3D virtual model 206 in response to virtual force 706 to simulate deformation of the real anatomical object 208 in response to physical force 214. Virtual model simulation system 202 may be configured to determine the additional set of displacement values by determining a displacement of one or more of nodes 702 due to virtual force 706. For example, displacement of nodes 702 may be determined based on a measure of virtual force 706 and material property values 210 of 3D virtual model 206 (e.g., based on Hooke's law). The positions of nodes 702 may be updated based on the determined additional set of displacement values. For illustrative purposes, FIG. 8 shows that virtual model simulation system 202 may move, based on the determined additional set of displacement values, a second node 702-1 of 3D virtual model 206 from a first position p₁ to a second position p₂ over the amount of time.

In instances where the location of a surface vertex of 3D virtual model 206 may not closely match to the location of a corresponding surface point of anatomical object 208, virtual model simulation system 202 may be configured to adjust one or more material property values 210 until the additional set of displacement values is within a threshold amount of the set of displacement values representative of the displacement of one or more of the surface points of anatomical object 208. For example, second node 702-1 of 3D virtual model 206 may correspond to first node 402-1 of anatomical object 208. After the application of physical force 214 and virtual force 706, the position (e.g., p₂) of second node 702-1 may be spaced a distance away from the location (e.g., 12) of first node 402-1. Virtual model simulation system 202 may adjust one or more material property values 210 of 3D virtual model 206 until the distance between position (e.g., p₂) of second node 702-1 is within a threshold amount from the location (e.g., 12) of first node 402-1. This may allow the displacement of second plurality of nodes 702 to correspond to the displacement of first plurality of nodes 402 such that the deformation of 3D virtual model 206 may mimic the deformation of anatomical object 208. Still other suitable methods for determining material property values 210 may be used.

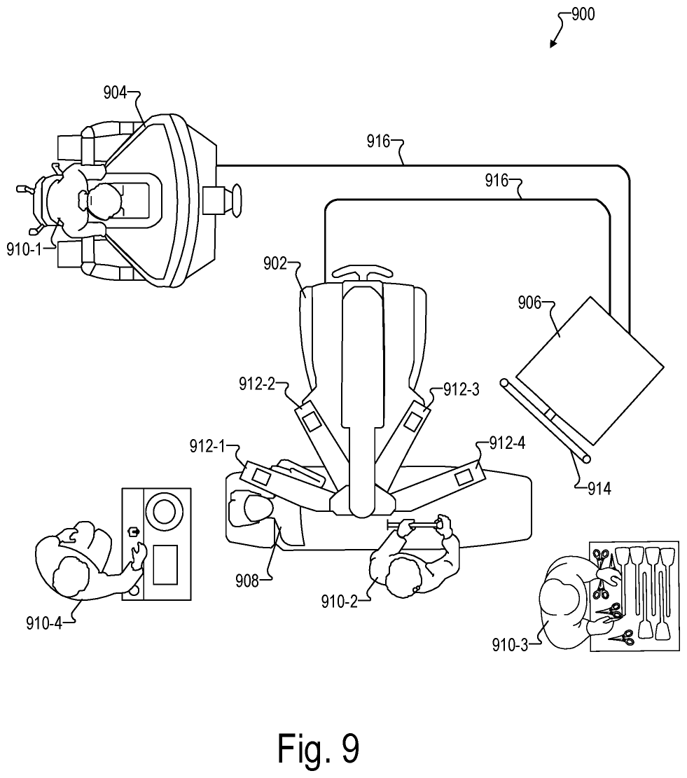

As has been described, virtual model simulation system 202, imaging device 204, and/or physical tool 212 may be associated in certain examples with a computer-assisted medical system used to perform a medical procedure on a body. To illustrate, FIG. 9 shows an illustrative computer-assisted medical system 900 that may be used to perform various types of medical procedures including surgical and/or non-surgical procedures.

As shown, computer-assisted medical system 900 may include a manipulator assembly 902 (a manipulator cart is shown in FIG. 9), a user control apparatus 904, and an auxiliary apparatus 906, all of which are communicatively coupled to each other. Computer-assisted medical system 900 may be utilized by a medical team to perform a computer-assisted medical procedure or other similar operation on a body of a patient 908 or on any other body as may serve a particular implementation. As shown, the medical team may include a first user 910-1 (such as a surgeon for a surgical procedure), a second user 910-2 (such as a patient-side assistant), a third user 910-3 (such as another assistant, a nurse, a trainee, etc.), and a fourth user 910-4 (such as an anesthesiologist for a surgical procedure), all of whom may be collectively referred to as users 910, and each of whom may control, interact with, or otherwise be a user of computer-assisted medical system 900. More, fewer, or alternative users may be present during a medical procedure as may serve a particular implementation. For example, team composition for different medical procedures, or for non-medical procedures, may differ and include users with different roles.

While FIG. 9 illustrates an ongoing minimally invasive medical procedure such as a minimally invasive surgical procedure, it will be understood that computer-assisted medical system 900 may similarly be used to perform open medical procedures or other types of operations. For example, operations such as exploratory imaging operations, mock medical procedures used for training purposes, and/or other operations may also be performed.

As shown in FIG. 9, manipulator assembly 902 may include one or more manipulator arms 912 (e.g., manipulator arms 912-1 through 912-4) to which one or more instruments may be coupled. The instruments may be used for a computer-assisted medical procedure on patient 908 (e.g., in a surgical example, by being at least partially inserted into patient 908 and manipulated within patient 908). While manipulator assembly 902 is depicted and described herein as including four manipulator arms 912, it will be recognized that manipulator assembly 902 may include a single manipulator arm 912 or any other number of manipulator arms as may serve a particular implementation. While the example of FIG. 9 illustrates manipulator arms 912 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to manipulator arms 912 shown in FIG. 9.

During the medical operation, user control apparatus 904 may be configured to facilitate teleoperational control by user 910-1 of manipulator arms 912 and instruments attached to manipulator arms 912. To this end, user control apparatus 904 may provide user 910-1 with images of an operational area associated with patient 908 as captured by an imaging device. To facilitate control of instruments, user control apparatus 904 may include a set of master controls. These master controls may be manipulated by user 910-1 to control movement of the manipulator arms 912 or any instruments coupled to manipulator arms 912.

Auxiliary apparatus 906 may include one or more computing devices configured to perform auxiliary functions in support of the medical procedure, such as providing insufflation, electrocautery energy, illumination or other energy for imaging devices, image processing, or coordinating components of computer-assisted medical system 900. In some examples, auxiliary apparatus 906 may be configured with a display monitor 914 configured to display one or more user interfaces, or graphical or textual information in support of the medical procedure. In some instances, display monitor 914 may be implemented by a touchscreen display and provide user input functionality. Augmented content provided by a region-based augmentation system may be similar, or differ from, content associated with display monitor 914 or one or more display devices in the operation area (not shown).

Manipulator assembly 902, user control apparatus 904, and auxiliary apparatus 906 may be communicatively coupled one to another in any suitable manner. For example, as shown in FIG. 9, manipulator assembly 902, user control apparatus 904, and auxiliary apparatus 906 may be communicatively coupled by way of control lines 916, which may represent any wired or wireless communication link as may serve a particular implementation. To this end, manipulator assembly 902, user control apparatus 904, and auxiliary apparatus 906 may each include one or more wired or wireless communication interfaces, such as one or more local area network interfaces, Wi-Fi network interfaces, cellular interfaces, and so forth.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

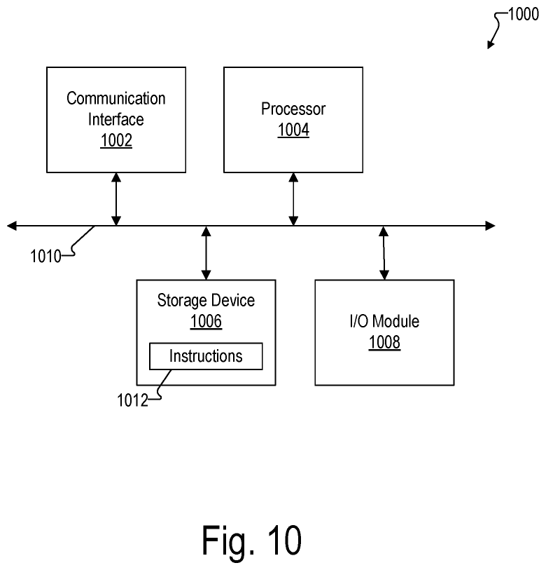

FIG. 10 shows an illustrative computing device 1000 that may be specifically configured to perform one or more of the processes described herein. Any of the systems, computing devices, and/or other components described herein may be implemented by computing device 1000.

As shown in FIG. 10, computing device 1000 may include a communication interface 1002, a processor 1004, a storage device 1006, and an input/output (“I/O”) module 1008 communicatively connected one to another via a communication infrastructure 1010. While an illustrative computing device 1000 is shown in FIG. 10, the components illustrated in FIG. 10 are not intended to be limiting. Additional or alternative components may be used in other embodiments. Components of computing device 1000 shown in FIG. 10 will now be described in additional detail.

Communication interface 1002 may be configured to communicate with one or more computing devices. Examples of communication interface 1002 include, without limitation, a wired network interface (such as a network interface card), a wireless network interface (such as a wireless network interface card), a modem, an audio/video connection, and any other suitable interface.

Processor 1004 generally represents any type or form of processing unit capable of processing data and/or interpreting, executing, and/or directing execution of one or more of the instructions, processes, and/or operations described herein. Processor 1004 may perform operations by executing computer-executable instructions 1012 (e.g., an application, software, code, and/or other executable data instance) stored in storage device 1006.

Storage device 1006 may include one or more data storage media, devices, or configurations and may employ any type, form, and combination of data storage media and/or device. For example, storage device 1006 may include, but is not limited to, any combination of the non-volatile media and/or volatile media described herein. Electronic data, including data described herein, may be temporarily and/or permanently stored in storage device 1006. For example, data representative of computer-executable instructions 1012 configured to direct processor 1004 to perform any of the operations described herein may be stored within storage device 1006. In some examples, data may be arranged in one or more databases residing within storage device 1006.

I/O module 1008 may include one or more I/O modules configured to receive user input and provide user output. I/O module 1008 may include any hardware, firmware, software, or combination thereof supportive of input and output capabilities. For example, I/O module 1008 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1008 is configured to provide graphical data to a display for presentation to a user. The graphical data may be representative of one or more graphical user interfaces and/or any other graphical content as may serve a particular implementation.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
a processor communicatively coupled to the memory and configured to execute the instructions to:
identify surface points on one or more anatomical objects;
determine a set of displacement values representative of a displacement of one or more of the surface points of the one or more anatomical objects caused by a physical force being applied by a physical tool to the one or more anatomical objects; and
define, based on the set of displacement values and data representative of the physical force, one or more material property values for a three-dimensional (3D) virtual model of the one or more anatomical objects, the one or more material property values representative of a material property of the one or more anatomical objects.

2. The system of claim 1, wherein the one or more material property values representative of the material property of the one or more anatomical objects define how the 3D virtual model deforms in response to manipulation of the 3D virtual model.

3. The system of claim 1, wherein the determining the set of displacement values comprises:
generating a point cloud having a plurality of nodes representative of the surface points on the one or more anatomical objects; and
tracking movement of the plurality of nodes over time while the physical force is applied to the one or more anatomical objects.

4. The system of claim 3, wherein the tracking movement of the plurality of nodes comprises determining a depth of the plurality of nodes relative to an initial position of the plurality of nodes prior to application of the physical force.

5. The system of claim 4, wherein the identifying surface points on the one or more anatomical objects is based on stereoscopic images of the one or more anatomical objects that are captured by an imaging device, and wherein determining the depth of the plurality of nodes comprises processing the stereoscopic images.

6. The system of claim 4, wherein the determining the depth of the plurality of nodes comprises generating, based on depth measurements obtained by a depth sensor, a depth map of the plurality of nodes.

7. The system of claim 1, wherein the data representative of the physical force comprises a measure of the physical force based on kinematic data representative of movement of the physical tool over time, the kinematic data generated by a computer-assisted medical system communicatively coupled with the physical tool.

8. The system of claim 1, wherein the data representative of the physical force comprises a measure of the physical force based on force data received by a force sensor communicatively coupled with the physical tool.

9. The system of claim 1, wherein the data representative of the physical force comprises one or more physical properties of the physical tool.

10. The system of claim 1, wherein the processor is further configured to execute the instructions to perform a registration of the 3D virtual model with a 3D model derived from one or more video images depicting the one or more anatomical objects.

11. The system of claim 10, wherein the processor is further configured to execute the instructions to:
identify, based on the registration, surface vertices on a surface of the 3D virtual model that correspond to the surface points on the one or more anatomical objects, the material properties of the 3D virtual model affecting how the surface vertices are displaced in response to manipulation of the 3D virtual model;
apply, to the 3D virtual model, a virtual force that simulates the physical force; and
determine an additional set of displacement values representative of a displacement of one or more of the surface vertices of the 3D virtual model caused by the virtual force.

12. The system of claim 11, wherein the virtual force is applied by a virtual instrument that simulates one or more physical properties of the physical tool.

13. The system of claim 11, wherein the determining the additional set of displacement values comprises:
generating a point cloud having a plurality of nodes representative of the surface vertices of the 3D virtual model; and
tracking movement of the plurality of nodes over time while the virtual force is applied to the 3D virtual model.

14. The system of claim 11, wherein the defining of the one or more material property values comprises adjusting one or more material property values until the additional set of displacement values is within a threshold amount of the set of displacement values representative of the displacement of one or more of the surface points of the one or more anatomical objects.

15. The system of claim 14, wherein the one or more of the material property values of the 3D virtual model are adjusted from one or more baseline material property values.

16. The system of claim 1, wherein the processor is further configured to execute the instructions to generate the 3D virtual model based on one or more preoperative images of the one or more anatomical objects.

17. The system of claim 16, wherein the generating the 3D virtual model comprises deriving a volumetric mesh model based on the one or more preoperative images of the one or more anatomical objects.

18. The system of claim 1, wherein the processor is further configured to execute the instructions to use the 3D virtual model during a simulation session in which a user interacts with the 3D virtual model to simulate physical interaction with the one or more anatomical objects.

19. A method comprising:
identifying, by at least one computing device, surface points on one or more anatomical objects;
determining, by the at least one computing device, a set of displacement values representative of a displacement of one or more of the surface points of the one or more anatomical objects caused by a physical force being applied by a physical tool to the one or more anatomical objects; and
defining, by the at least one computing device based on the set of displacement values and data representative of the physical force, one or more material property values for a three-dimensional (3D) virtual model of the one or more anatomical objects, the one or more material property values representative of a material property of the one or more anatomical objects.

20-29. (canceled)

30. A non-transitory computer-readable medium storing instructions that, when executed, direct a processor of a computing device to:
identify surface points on one or more anatomical objects;
determine a set of displacement values representative of a displacement of one or more of the surface points of the one or more anatomical objects caused by a physical force being applied by a physical tool to the one or more anatomical objects; and
define, based on the set of displacement values and data representative of the physical force, one or more material property values for a three-dimensional (3D) virtual model of the one or more anatomical objects, the one or more material property values representative of a material property of the one or more anatomical objects.
